(19) United States
(12) Patent Application Publication
(10) Pub. No.: US 2010/0215697 A1
(43) Pub. Date: Aug. 26, 2010

(54) METHODS AND MATERIALS FOR MAKING AND USING VACCINES

(76) Inventors: Stanimir Vuk-Pavlovic, Rochester, MN (US); Gaylord J. Knutson, Rochester, MN (US)

Correspondence Address: FISH & RICHARDSON P.C., PO BOX 1022, MINNEAPOLIS, MN, 55440-1022, US

(21) Appl. No.: 12/712,828

(22) Filed: Feb. 25, 2010

Related U.S. Application Data
(60) Provisional application No. 61/155,756, filed on Feb. 26, 2009.

(51) Int. Cl. A61K 39/00; A61P 35/00

(52) U.S. Cl. 424/277.1

(57) ABSTRACT

This document relates to methods and materials for making and using vaccines. For example, vaccine preparations (e.g., whole cell vaccines and cell lysate vaccines) that can be used to treat cancer (e.g., human ovarian cancer) are provided.

### CROSS-REFERENCE TO RELATED APPLICATIONS

This application claims priority to U.S. Provisional Application Ser. No. 61/155,756, filed Feb. 26, 2009. The disclosure of the prior application is considered part of (and is incorporated by reference in) the disclosure of this application.

### BACKGROUND

1. Technical Field

This document relates to methods and materials for making and using vaccines. For example, this document provides vaccines (e.g., whole cell vaccines and cell lysate vaccines) that can be used to treat cancer (e.g., human ovarian cancer).

2. Background Information

Cancer is a serious illness that affects many people every year. There are over one million new cancer cases and over 500,000 deaths per year from cancer in the United States. The high mortality rate from cancer highlights the need for improved cancer detection and treatment.

### SUMMARY

This document relates to methods and materials for making and using vaccines. For example, this document provides vaccines (e.g., whole cell vaccines and cell lysate vaccines) that can be used to treat cancer (e.g., human ovarian cancer). As described herein, cancer cells cultured under partial oxygen pressures (pO₂) less than in ambient air (e.g., less than 21.2 kPa at sea level or ambient pressure at the particular altitude above sea level) or ambient air containing carbon dioxide, usually 5.0 percent, or pO₂ greater than 21.2 kPa at sea level (or ambient pressure at the particular altitude above sea level) can have a macromolecular expression profile that is different than that observed in the same cells cultured under an pO₂ of 21.2 kPa at sea level or ambient pressure at the particular altitude above sea level.

In general, one aspect of this document features a method for making a whole cell vaccine preparation. The method comprises culturing cells at an oxygen pressure of between 0.5 kPa and 10 kPa for a period of time of at least four hours, and obtaining the cells to produce the whole cell vaccine preparation. The cells can be ovarian cancer cells. The cells can be OV17, OV167, or OV207 cells. The oxygen pressure can be between 1 kPa and 5 kPa. The period of time can be at least 12 hours.

In another aspect, this document features a method for making a cell lysate vaccine preparation. The method comprises lysing cells cultured at an oxygen pressure of between 0.5 kPa and 10 kPa for a period of time of at least four hours, and combining the resulting lysate with a pharmaceutically-accepted carrier to produce the cell lysate vaccine preparation. The cells can be ovarian cancer cells. The cells can be OV17, OV167, or OV207 cells. The oxygen pressure can be between 1 kPa and 5 kPa. The period of time can be at least eight hours. The pharmaceutically-accepted carrier can be alum.

In another aspect, this document features a whole cell vaccine preparation comprising cancer cells cultured at an oxygen pressure of between 0.5 kPa and 10 kPa for a period of time of at least four hours. The cancer cells can be ovarian cancer cells. The cells can be OV17, OV167, or OV207 cells. The oxygen pressure can be between 1 kPa and 5 kPa. The period of time can be at least eight hours. The vaccine preparation can comprise a pharmaceutically-accepted carrier.

In another aspect, this document features a cell lysate vaccine preparation comprising a lysate of cancer cells cultured at an oxygen pressure of between 0.5 kPa and 10 kPa for a period of time of at least four hours. The cancer cells can be ovarian cancer cells. The cells can be OV17, OV167, or OV207 cells. The oxygen pressure can be between 1 kPa and 5 kPa. The period of time can be at least eight hours. The vaccine preparation can comprise a pharmaceutically-accepted carrier. The pharmaceutically-accepted carrier can be alum.

In another aspect, this document features a method for vaccinating a mammal having cancer. The method comprises administering, to the mammal, a whole cell vaccine preparation or cell lysate vaccine preparation made using cancer cells cultured at an oxygen pressure of between 0.5 kPa and 10 kPa for a period of time of at least four hours. The cancer cells can be ovarian cancer cells. The cells can be OV17, OV167, or OV207 cells. The oxygen pressure can be between 1 kPa and 5 kPa. The period of time can be at least eight hours. The vaccine preparation can comprise a pharmaceutically-accepted carrier. The pharmaceutically-accepted carrier can be alum. The mammal can be a human. The cancer cells can be obtained from the mammal prior to being cultured.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF THE DRAWINGS

FIG. 1. A. LnCap prostate cancer cells (10,000 cells/cm²) were cultured at pO₂=2 kPa (right panel) or 20 kPa (left panel). After three days, cells were viewed with the aid of a phase contrast light microscope at 200× magnification. B. Live cells were counted in triplicate wells using trypan blue exclusion to differentiate dead cells. C. The rate of VEGF secretion in hypoxia-grown cells peaked earlier and higher than in normoxia-grown cells. D. 2D-SDS electrophoresis (2-DE) of lysed LnCap cells cultured at pO₂=2 kPa was performed. First dimension: pH 3-10; Second dimension: 8-16 percent SDS-PAGE gel. Rectangular marks: 86 spots identified as twice or more intense after 3 days at pO₂=2 kPa than at 20 kPa. E. New autoantibodies (drawn box or circles) against LnCaP cells grown at pO₂=2 kPa in diagnosed CaP patients were detected. First dimension: pH 5-8; Second dimension: 8-16 percent SDS-PAGE gel, transferred to a nylon membrane, incubated with pooled plasma (1:300) from newly diagnosed CaP patients (n=18) or age matched controls (n=12). Following incubation with mouse anti-human IgG (1:6000) and with goat anti-mouse Ig HRP (1:3000), immunodetection was achieved by chemiluminescence. * p<0.05, relative to pO₂=20 kPa; # p<0.05, relative to cell number at 18 hours of incubation at the same pO₂ value.

### DETAILED DESCRIPTION

This document provides methods and materials for making and using vaccines. For example, this document provides vaccine preparations (e.g., whole cell vaccine and cell lysate vaccine preparations) that can be used to treat cancer (e.g., human ovarian cancer).

The vaccine preparations provided herein can be in the form of whole cell vaccine preparations or vaccine preparations containing products obtained from cells (e.g., a cell lysate vaccine preparation). In general, the vaccine preparations provided herein can be used to induce an immune response against any type of cancer including, without limitation, ovarian, prostate, colon, breast, kidney, liver, lung and other cancers. For example, the vaccine preparations provided herein can be designed to contain human ovarian cancer cells or a lysate of human ovarian cancer cells. Examples of ovarian cancer cells that can be cultured as described herein to make a vaccine preparation include, without limitation, the cell lines designated OV17, OV167, OV207, and other ovarian cancer cell lines. Table 1 provides a list of cancer cell lines that can be cultured as described herein to prepare a vaccine preparation to treat the indicated cancer. The vaccine preparations provided herein can be used to treat cancer in any type of mammal including, without limitation, humans, cows, pigs, monkeys, dogs, cats, horses, and other mammals.

TABLE 1

| Cancer | Cell Line Designation |
|---|---|
| Acute monocyte leukemia | AML-193 |
| Adenocarcinoma primary, unknown, metastatic | Hs 696 |
| to bone-sacrum | |
| Adenocarcinoma, breast | MDA-MB-415 |
| Adenocarcinoma, breast | MDA-MB-436 |
| Adenocarcinoma, breast | MDA-MB-468 |
| Adenocarcinoma, breast, malignant pleural | SK-BR-3 |
| effusion | |
| Adenocarcinoma, breast, metastasis to brain | MDA-MB-361 |
| Adenocarcinoma, colon | Caco-2 |
| Adenocarcinoma, colon, ascites | SK-CO-1 |
| Adenocarcinoma, colon, moderately well- | HT-29 |
| differentiated grade II | |
| Adenocarcinoma, duodenum | HuTu 80 |
| Adenocarcinoma, kidney | A-704 |
| Adenocarcinoma, kidney | SW 839 |
| Adenocarcinoma, liver, ascites | SK-HEP-1 |
| Adenocarcinoma, lung consistent with poorly | SK-LU-1 |
| differentiated. grade III | |
| Adenocarcinoma, lung, pleural effusion | Calu-3 |
| Adenocarcinoma, metastatic to pelvis | Hs 700T |
| Adenocarcinoma, ovary, consistent with | Caov-3 |
| primary | |
| Adenocarcinoma, ovary, malignant ascites | SK-OV-3 |
| Adenocarcinoma, ovary, metastasis to | Caov-4 |
| subserosa of fallopian tube | |
| Adenocarcinoma, pancreas | Capan-2 |
| Adenocarcinoma, pancreas, metastasis to liver | Capan-1 |
| Adenocarcinoma., lung | NCI-H676B |
| Adenocarcinoma., ovary | SW 626 |
| Adenosquamous carcinoma, lung | NCI-H596 |
| Amelanotic melanoma, metastatic to lymph | Hs 695T |
| node | |
| Anaplastic carcinoma, probably lung | Calu-6 |
| Anaplastic osteosarcoma versus Swing | SK-ES-1 |
| sarcoma, bone | |
| Astrocytoma | SW 1088 |
| Astrocytoma | SW 1783 |
| Axilla synovial sarcoma | SW 982 |
| Bladder, normal fetus | FHs 738B1 |
| Breast | HBL-100 |
| Breast adenocarcinoma, pleural effusion | MCF7 |
| Breast, ductal carcinoma, pleural I effusion | MDA-MB-134-V |
| Breast, ductal carcinoma, pleural II effusion | MDA-MB-175-V |
| Breast, medulla, carcinoma, pleural effusion | MDA-MD-157 |
| Breast, normal | Hs 578Bst |
| Bronchogenic carcinoma, subcutaneous | CHaGo K-l |
| metastasis, human | |
| Burkitt lymphoma, ascites | P3HR-1 |
| Burkitt lymphoma, ovary | EB2 |
| Burkitt lymphoma, upper maxilia | ED1 |
| Carcinoma, bladder, primary | 5637 |
| Carcinoma, breast | BT-20 |
| Carcinoma, breast | MDA-MB-330 |
| Carcinoma, breast | MDA-MB-453 |
| Carcinoma, cervix | C-33A |
| Carcinoma, cervix, metastasis to lymph node | HT-3 |
| Carcinoma, kidney | A-498 |
| Carcinoma, lung | A-427 |
| Carcinoma, pancreas, metastatic to lymph node | Hs 766T |
| Carcinoma, prostate, metastasis to brain | DU 145 |
| Carcinoma, stomach, metastatic to left leg | Hs 746T |
| Carcinoma, vulva, lymph node metastasis | SW 962 |
| Chondrosarcoma, humerus | SW 1353 |
| Choriocarcinoma | JEG-3 |
| Choriocarcinoma, placenta | JAR |
| Clear cell carcinoma, consistent with renal | Caki-2 |
| primary | |
| Clear cell carcinoma, consistent with renal | Caki-1 |
| primary, metastasis to skin | |
| Ductal carcinoma, breast | BT-474 |
| Ductal carcinoma, breast | BT-483 |
| Ductal carcinoma, breast | 8T-549 |
| Ductal carcinoma, breast | Hs 578T |
| Ductal carcinoma, breast | MDA-MB-435S |
| Ductal carcinoma, breast, pleural effusion | T-47D |
| Embryonal carcinoma, malignancy consistent | Tera-1 |
| with metastasis to lung | |
| Embryonal carcinoma, malignancy consistent | Tera-2 |
| with metastasis to lung | |
| Embryonal carcinoma, testis, metastasis to | Cate-1B |
| lymph node | |
| Endometrial adenocarcinoma | HEC-1-A |
| Endometrial adenocarcinoma | HEC-1-B |
| Endometrial adenocarcinoma, metastatic | AN3 CA |
| Epidermoid carcinoma grade III, lung, | Calu-1 |
| metastasis to pleura | |
| Epidermoid carcinoma, cervix, metastasis to | MS751 |
| lymph node | |
| Epidermoid carcinoma, cervix, metastasis to | ME-180 |
| omentum | |
| Epidermoid carcinoma, submaxillary gland | A-253 |
| Ewing's sarcoma | RD-ES |
| Fibrosarcoma | SW684 |
| Fibrosarcoma, metastatic to lung | Hs 913T |
| Gastric carcinoma | KATO III |
| Glioblastoma | U-118 MG |
| Glioblastoma | U-138 MG |
| Glioblastoma, astrocytoma, grade III | U-87 MG |
| Glioblastoma, astrocytoma, grade III | U-373 MG |
| Glioma | Hs 683 |
| Large cell carcinoma, lung | NCI-H661 |
| Large cell carcinoma, lung | NCI-H460 |
| Leiomyosarcoma, vulva, primary | SK-LNS-1 |
| Leukemia biphenotype | MV4-11 |
| Liposarcoma | SW 872 |
| Lung, normal fetus | FHs 738Lu |
| Lymphoid, Hodgkin's disease | Hs 445 |
| Lymphoma, cervical | Hs 602 |
| Malignant melanoma | SK-MEL-28 |
| Malignant melanoma | SK-MEL-31 |
| Malignant melanoma, metastasis to axillary | SK-MEL-5 |
| node | |
| Malignant melanoma, metastasis to lung | Malme-3M |
| Malignant melanoma, metastasis to lymph | RPMI-7951 |
| node | |
| Malignant melanoma, metastasis to lymph | SK-MEL-3 |
| node | |
| Malignant melanoma, metastasis to lymphatic | SK-MEL-1 |
| system | |
| Malignant melanoma, metastasis to node | SK-MEL-24 |
| Malignant melanoma, metastasis to skin of | SK•MEL-2 |
| thigh | |
| Malignant melanoma, metastasis to | HT-I44 |
| subcutaneous tissue | |
| Medulloblastoma | D283 Med |
| Medulloblastoma | Daoy |
| Medulloblastoma | D341 Med |
| Melanoma | MEL-175 |
| Melanoma | MEL-290 |
| Melanoma | Sk-Mel28 |
| Melanoma cells | HLA-A*0201 |
| Melanoma, metastatic to lymph node | Hs 294T |
| Metastatic cutaneous nodule, breast carcinoma | DU4475 |
| Neuroblastoma, metastasis to bone marrow | SK-N-SH |
| Neuroblastoma, metastasis to supra-orbital area | SK-N-MC |
| Neuroglioma, brain | H4 |
| Osteogenic sarcoma, bone primary | U-2 OS |
| Osteogenic sarcoma, primary | Saos-2 |
| Ovary, adenocarcinoma | NlH:OYCAR-3 |
| Ovary, adenocarcinoma, endometrioid | OV17 |
| Ovary, adenocarcinoma, serous | OV167 |
| Ovary, adenocarcinoma, clear cell | OV207 |
| Papillary adenocarcinoma, lung | NCI-H820 |
| Papillary adenocarcinoma, lung | NCI-H441 |
| Retinoblastoma | Y79 |
| Retinoblastoma | WERI-Rb-1 |
| Rhabdomyosarcoma | A-204 |
| Rhabdomyosarcoma. left leg | Hs 729 |
| Skin fibroblast | Malme-3 |
| Small cell carcinoma, extra-pulmonary origin, | NCI-HS10A |
| metastatic | |
| Small cell carcinoma, lung | NCI-H69 |
| Small cell carcinoma, lung | NCI-H128 |
| Small cell carcinoma, lung | NCI-H446 |
| Small cell carcinoma, lung | NCI-H209 |
| Small cell carcinoma, lung | NCI-H146 |
| Small cell carcinoma, lung | NCI-H345 |
| Small cell carcinoma. lung | NCI-H82 |
| Squamous carcinoma, bladder | ScaBER |
| Squamous carcinoma, cervix | SiHa |
| Squamous carcinoma, lung, pleural effusion | SK-MES-1 |
| Squamous cell carcinoma, pharynx | FaDu |
| Squamous cell carcinoma, vulva | SW 954 |
| Squamous cell carcinoma. lung | SW 900 |
| Squamous cell carcinoma., lung | NCI-H520 |
| T-cell lymphoma | H9 |
| Thymus, normal | Hs 67 |
| Thyroid carcinoma | SW579 |
| Transitional-cell carcinoma, bladder | J82 |
| Transitional-cell carcinoma, bladder | T24 |
| Transitional-cell carcinoma, bladder, primary | TCCSUP |
| grade IV | |
| Transitional-cell papilloma, bladder | RT4 |
| Uterine, mixed mesodermal tumor, consistent | SK-UT-1 |
| with Leiomyosarcoma grade III | |
| Whole embryo, normal | FHs l73We |
| Wilms' tumor, pleural effusion | SK-NEP-1 |

The cells used to make a vaccine preparation provided herein can be cultured at a particular stable pO₂ level between 0.5 and 21.2 kPa (e.g., between 0.5 and 20 kPa, between 1 and 20 kPa, between 5 and 20 kPa, between 0.5 and 10 kPa, between 0.5 and 5 kPa, between 0.5 and 2 kPa, between 1 and 10 kPa, between 1 and 5 kPa, or between 1 and 2 kPa) for a period of time greater than three hours (e.g., greater than 4, 5, 6, 7, 8, 9, 10, 11, 12, 18, 24, 36, 48, or 96 hours). In some cases, the cells can be cultured at 0.5 kPa, 2.0 kPa, 5 kPa, or 10 kPa. In some cases, the cells used to make a vaccine preparation provided herein can be cultured at an pO₂ that is between 0.5 and 15 kPa for a period of time between 12 hours and four weeks (e.g., between 12 hours and two weeks, between 12 hours and one week, between 12 hours and three days, between 12 hours and 48 hours, between 24 hours and four weeks, between 24 hours and two weeks, or between 24 hours and one week).

The vaccine preparations provided herein can include whole cells or portions of cells (e.g., a cell lysate) in combination with a pharmaceutically acceptable carrier. Examples of pharmaceutically acceptable carriers include, without limitation, alum and biodegradable three-dimensional macromolecular matrices. In some cases, a vaccine preparation provided herein can include whole cells or portions of cells that were cultured in serum-free conditions for at least the duration of one cell doubling.

The vaccine preparations provided herein can include whole cells or portion of cells (e.g., a cell lysate) combined ex vivo with autologous or haploidentical of allogeneic antigen presenting cells (APCs) that internalize them, process and present as epitopes. Examples of acceptable APCs include, without limitation, dendritic cells. In some cases, a vaccine preparation provided herein can include whole cells or portions of cells that were cultured in serum-free conditions for at least the duration of one cell doubling.

The vaccine preparations provided herein can include portions of cells (e.g., a cell lysate) combined ex vivo with bioartificial antigen-presenting carriers. Examples of bioartificial antigen-presenting carriers include three-dimensional particles, including but not limited to, nanoparticles and microsomes without or combined with antigen-presenting molecules and/or co-stimulatory molecules and/or releasable cytokines and/or chemokines In some cases, a vaccine preparation provided herein can include portions of cells that were cultured in serum-free conditions for at least the duration of one cell doubling.

The vaccine preparations provided herein can be used as stand alone vaccines or can be used in combination with other vaccines (e.g., one or more polypeptides derived from the sequence of antigens characteristic for the tissue of cancer being treated, e.g., MUC16 or CA125 for ovarian cancer).

In some cases, a vaccine preparation provided herein can be formulated with an adjuvant. An adjuvant can be an immunological compound that can enhance an immune response against a particular antigen preparation such as a whole cell preparation or cell lysate preparation provided herein. Suitable adjuvants include, without limitation, a thalidomide derivative (e.g., revlimid), Bacille Calmette-Guerin (BCG), monophosphoryl lipid A and its derivatives, and alum as well as other aluminum-based compounds (e.g., Al₂O₃) that can be obtained from various commercial suppliers. In some cases, MN51 can be combined with a vaccine preparation provided herein to form a composition that elicits an immune response when administered to a mammal. MN51 contains mannide oleate (MONTANIDE® 80, also known as anhydro mannitol octadecenoate) in mineral oil solution. Other adjuvants include immuno-stimulating complexes (ISCOMs) that can contain such components as cholesterol and saponins ISCOM matrices can be prepared and conjugated to Cu²⁺.

This document also provides methods for preparing a vaccine preparation provided herein. Such methods can involve culturing cancer cells under pO₂ (e.g., controlled pO₂) less than the pO₂ in air or in air enriched with carbon dioxide, usually 5 percent, at ambient pressure at the particular elevation (such as 1-5 kPa) or greater than the pO₂ in air or in air enriched with carbon dioxide, usually 5 percent, at ambient pressure at the particular elevation (such as 50-100 kPa) for a period of time as described herein. Once cultured, the cells can be harvested and used as a whole cell vaccine preparation or can be lysed to create a cell lysate vaccine. In some cases, particular portions or fractions of the cells can be used to make a vaccine preparation. In some cases, antigen-presenting cells, bioartificial antigen-presenting carriers, an adjuvant or a pharmaceutically acceptable carrier can be included. The combining step can be achieved by any appropriate method, including, for example, incubation, stirring, shaking, vortexing, or passing back and forth through a needle attached to a syringe.

It is noted that the compositions can be prepared in batch, such that enough unit doses are obtained for multiple injections (e.g., injections into multiple mammals or multiple injections into the same mammal). A “unit dose” of a composition provided herein refers to the amount of a composition administered to a mammal at one time. A unit dose of the compositions provided herein can contain any amount of cellular material. For example, a unit dose of a composition can contain between 1×10⁶ cells and 100×10⁶ cells or the amounts of lysate prepared from equivalent numbers of cells. Alternatively, doses can be defined as ranging from about 0.1 μg and about 1.0 g (e.g., 1 μg, 10 μg, 15 μg, 25 μg, 30 μg, 50 μg, 100 μg, 250 μg, 280 μg, 300 μg, 500 μg, 750 μg, 1 mg, 10 mg, 15 mg, 25 mg, 30 mg, 50 mg, 100 mg, 250 mg, 280 mg, 300 mg, 500 mg, 750 mg, or more) of macromolecular material from cultured cells.

Methods for inducing a particular anti-cancer immune response in a mammal (e.g., a mouse, a rat, a cat, a dog, a horse, a cow, a non-human primate such as a cynomolgus monkey, or a human) include, without limitation, administering to a mammal an amount of a vaccine preparation provided herein that is effective for producing an anti-cancer response.

The vaccine preparations provided herein can be administered using any appropriate method. Administration can be, for example, by subcutaneous, intrathecal, intraventricular, intramuscular, or intraperitoneal injection, or by intravenous drip. Administration can be rapid (e.g., by injection) or can occur over a period of time (e.g., by slow infusion).

The invention will be further described in the following examples, which do not limit the scope of the invention described in the claims.

### Examples

### Example 1

### Oxygen Regulated Macromolecule Expression by Ovarian Carcinoma Cells

Oxygen pressure in standard cell culture is about 20 kPa. To determine if pO₂ affects ovarian cancer (OvCa) cells developed for an injectable vaccine, human OvCa cells OV17, OV167, or OV207 were cultured at an pO₂ of either 1-2 kPa, 20 kPa, or 90 kPa. The cultures were analyzed for the following: proliferation, secretion of vascular endothelial growth factor (VEGF, a hallmark of OvCa), level of intracellular hypoxia-inducible factor 1α (HIF-1α), and cellular proteome. VEGF concentration in the media increased with time at all pO₂ values (p<0.01). An pO₂ of 1-2 kPa enhanced the rate of VEGF secretion in all cells. An pO₂ of 90 kPa abolished proliferation (p=0.0008), but unexpectedly boosted VEGF secretion (p=0.0258). HIF-1α levels were compared in cells cultured at an pO₂ of 20 kPa and cells cultured at an pO₂ of 1-2 kPa or at an pO₂ of 90 kPa for 18 to 24 hours. HIF-1α levels in cells cultured at an pO₂ of 1-2 kPa were higher than that observed in cells cultured at an pO₂ of 20 kPa. Interestingly, cells cultured at an pO₂ of 90 kPa also expressed high HIF-1α levels paralleling high VEGF secretion. Adherent OvCa cells responded both to hypoxia (e.g., an pO₂ of 1-2 kPa) and hyperoxia (e.g., an pO₂ of 90 kPa) by elevated levels of HIF-1α and VEGF.

About 1,400 spots were analyzed on 2D gels of OV167 cells grown at an pO₂ of either 2 kPa or 20 kPa. 919 spots (66%) matched in position. Of these, 192 spots changed intensity twofold, 51 spots fivefold, and 16 spots tenfold or more. Several spots were present only in cells cultured at an pO₂ of 2 kPa or an pO₂ of 20 kPa. These findings support the hypothesis that pO₂ levels modify protein expression patterns of cultured OvCa cells.

These results demonstrate that whole cell vaccines and cell lysate vaccines can be prepared using cells cultured under controlled pO₂ (e.g., an pO₂ less than 20 kPa such as 1-5 kPa or an pO₂ greater than 20 kPa such as 50-100 kPa).

### Example 2

### Whole Cell Vaccine Preparation

OV17, OV167, or OV207 cells are cultured from the vaccine-grade master cell banks at pO₂ of either 1-2 kPa or 90 kPa in a clinical (cGMP) grade cell culture medium containing cGMP-grade fetal bovine serum, or human serum, or synthetic supplements that support growth and/or viability of the cells. After the period required to complete at least one full cell cycle in culture, the cells are pooled, and harvested. The cells are irradiated prior to aliquotting to render them replication incompetent. The dose of irradiation used is 150 Gy. Samples of frozen cells are tested for sterility, presence of mycoplasma and lypolysaccharide, ability to proliferate and are subjected to other tests as required. The cells are released for administration after meeting all release criteria. Prior to injection, the cells of the three cell lines are thawed and mixed. The cells can be injected directly as the whole-cell vaccine. Alternatively, the cells can be incubated ex vivo with patient's own antigen-presenting cells (such as dendritic cells), allogeneic antigen-presenting cells, or biosynthetic antigen-presenting systems. Combinations of cancer vaccine cells and antigen-presenting cells can be further incubated with agents that will mature and/or modify the antigen-presenting cells.

### Example 3

### Cell Lysate Vaccine Preparation

The cells described in Example 2 are lysed. Methods for lysis can include one or more of the following: repeated freeze-thaw cycles, osmotic shock, radiation, heat, cold, pressure, grinding, sonication, drying, and detergents. Cell lysate is then incubated with the patient's own antigen-presenting cells, allogeneic antigen-presenting cells (such as dendritic cells), or biosynthetic antigen-presenting systems. Combinations of cancer vaccine cell lysates and antigen-presenting cells can be further incubated with agents that will mature and/or modify the antigen-presenting cells.

### Example 4

### Oxygen Regulated Macromolecule Expression by Prostate Carcinoma Cells

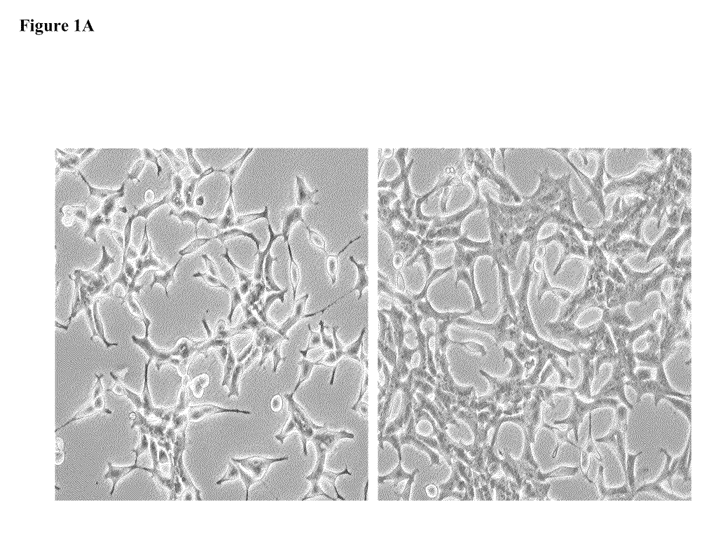

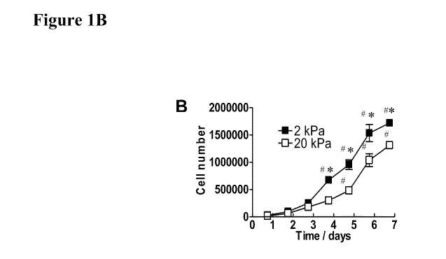

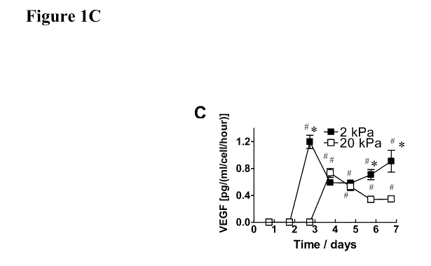

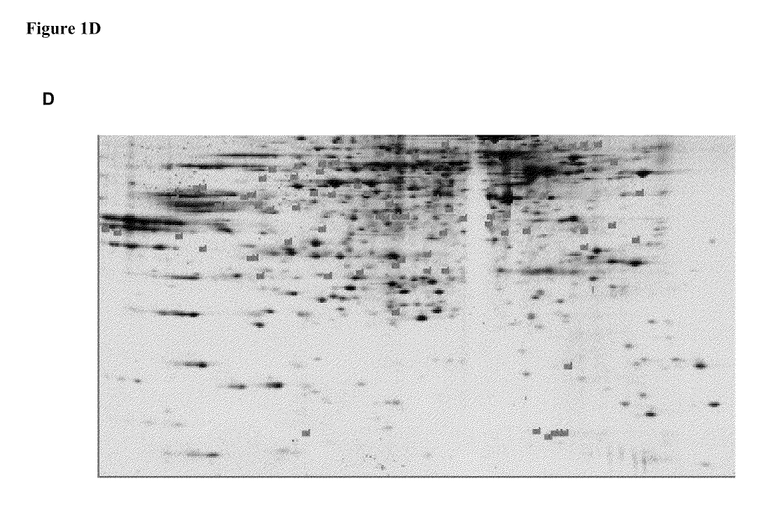

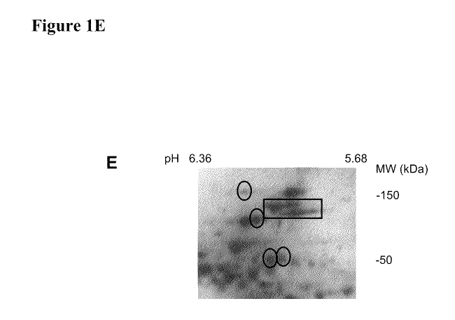

The effects of pO₂ on select biological properties of prostate cancer (PCa) cells in culture was studied. As a model, the well-known human LnCaP cells derived from a lymph node metastasis (Horoszewicz et al., Cancer Res., 43:1809-18 (1983)) were used. This cell line was established in 1977 and was deposited in the ATCC reference bank (see, e.g., ATCC Number CRL-1740). LnCaP cells express prostate specific antigen (PSA) and retain a functional androgen receptor pathway. In two-dimensional cultures, hypoxic LnCaP cells (pO₂=2 kPa) proliferated faster than cell cultured at standard cell culture conditions (FIGS. 1A and 1B). In addition, hypoxic cells secreted more VEGF (FIG. 1C), as described elsewhere (Ghafar et al., Prostate, 54:58-67 (2003)). A characterization of the proteome by 2-DE revealed 86 spots that differed in intensity and/or position between LnCaP cells grown at pO₂=2 kPa and LnCaP cells grown at 20 kPa (FIG. 1D).

To determine how LnCaP cells grown under hypocial compare to LnCaP cells grown normoxically with regard to raising and immune response in humans, the following was performed to determine if sera from PCa patients contained spontaneous antibodies cross-reactive with LnCaP cells. Two-dimensional electrophoresis of LnCaP cell lysate was performed, and the resolved proteins were transferred to the nylon membrane that was incubated with a PCa patient's plasma, washed, and incubated with the anti-human IgG antibody to detect the putative sites of human antibody binding. As a result, numerous LnCaP cell proteins were found to bind antibodies from PCa patients (FIG. 1E). When the number of spots identified by the patient plasma in LnCaP cells grown at low pO₂ was compared to those grown at 20 kPa, more spots were found in hypoxic cells (FIG. 1E). This results demonstrate that protein expression and the antigenic signature of cultured hypoxic cells can be more akin to that of tumor cells in situ as compared to normoxic cells. Overall, these results indicate that hypoxia profoundly affects growth, the proteome, and the antigenic signature of PCa cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making a cell lysate vaccine preparation, wherein said method comprises:
(a) lysing cells cultured at an oxygen pressure of between 0.5 kPa and 10 kPa for a period of time of at least four hours, and
(b) combining the resulting lysate with a pharmaceutically-accepted carrier to produce said cell lysate vaccine preparation.

2. The method of claim 1, wherein said cells are ovarian cancer cells.

3. The method of claim 1, wherein said cells are OV17, OV167, or OV207 cells.

4. The method of claim 1, wherein said oxygen pressure is between 1 kPa and 5 kPa.

5. The method of claim 1, wherein said period of time is at least eight hours.

6. The method of claim 1, wherein said pharmaceutically-accepted carrier is alum.

7. A cell lysate vaccine preparation comprising a lysate of cancer cells cultured at an oxygen pressure of between 0.5 kPa and 10 kPa for a period of time of at least four hours.

8. The method of claim 7, wherein said cancer cells are ovarian cancer cells.

9. The method of claim 7, wherein said cells are OV17, OV167, or OV207 cells.

10. The method of claim 7, wherein said oxygen pressure is between 1 kPa and 5 kPa.

11. The method of claim 7, wherein said period of time is at least eight hours.

12. The method of claim 7, wherein said vaccine preparation comprises a pharmaceutically-accepted carrier.

13. The method of claim 12, wherein said pharmaceutically-accepted carrier is alum.

14. A method for vaccinating a mammal having cancer, wherein said method comprises administering, to said mammal, a cell lysate vaccine preparation made using cancer cells cultured at an oxygen pressure of between 0.5 kPa and 10 kPa for a period of time of at least four hours.

15. The method of claim 14, wherein said cancer cells are ovarian cancer cells.

16. The method of claim 14, wherein said cells are OV17, OV167, or OV207 cells.

17. The method of claim 14, wherein said oxygen pressure is between 1 kPa and 5 kPa.

18. The method of claim 14, wherein said period of time is at least eight hours.

19. The method of claim 14, wherein said vaccine preparation comprises a pharmaceutically-accepted carrier.

20. The method of claim 14, wherein said pharmaceutically-accepted carrier is alum.

21. The method of claim 14, wherein said mammal is a human.

22. The method of claim 14, wherein said cancer cells were obtained from said mammal prior to being cultured.
